(19) United States
(12) Patent Application Publication
(10) Pub. No.: US 2022/0203313 A1
(43) Pub. Date: Jun. 30, 2022

(54) Pressurized Fluid Mixing Device

(71) Applicant: Zhi'an Ou, Guangdong (CN)

(72) Inventors: Zhi'an Ou, Guangdong (CN); Xueying Ou, Guangdong (CN)

(21) Appl. No.: 17/638,132

(22) PCT Filed: Sep. 10, 2019
(86) PCT No.: PCT/CN2019/104999

§ 371 Date: Feb. 24, 2022

(30) Foreign Application Priority Data
Aug. 30, 2019 (CN) 201910816033.6

(51) Int. Cl. B01F 25/435; B01F 25/431; B01F 35/93; B01J 19/00; B01J 19/24; F28F 3/12

(52) U.S. Cl. CPC B01F 25/435; B01F 25/4319; B01F 25/431971; B01F 35/93; B01F 2035/98; B01J 19/2415; B01J 19/244; F28F 3/12; B01F 2215/0431; B01J 19/0013

(57) ABSTRACT

A pressurized fluid mixing device is disclosed, including an inner casing and an outer casing. A first channel is arranged in the inner casing and includes one or more unit channels, adjacent unit channels of which are communicated with each other, flow blocking members are fixed on the unit channels, the inner casing is provided with one or more first inlets and one or more first outlets, a second channel is arranged in the outer casing, the outer casing is provided with one or more second inlets and one or more second outlets, and the inner casing is fixed on the second channel.

### CROSS-REFERENCE TO RELATED APPLICATIONS

This application is the United States national phase of International application number PCT/CN2019/104999, filed Sep. 10, 2019, and claims priority to Chinese patent application No. 201910816033.6 filed Aug. 30, 2019, the disclosures of which are hereby incorporated by reference in their entirety.

### BACKGROUND

### Technical Field

The disclosure relates to the technical field of food and chemical fluid mixing, in particular to a pressurized fluid mixing device.

### Technical Considerations

China is a big chemical country. Every year, a large number of enterprises and chemical plants need to mix or perform a mix reaction on a large number of fluids to synthesize required products. Application of a traditional tank-type mixing reactor is usually composed of feeding, heat transfer, transmission, stirring, sealing and other parts. Its bulky volume and large amount of single raw material addition lead to too long mixing reaction time and greatly reduced mixing efficiency. The added materials also contain flammable, explosive, toxic, media corrosion and other characteristics, which are extremely dangerous. Moreover, in order to stably control the reaction temperature of the mixture, it is usually necessary to additionally equip corresponding cooling and heat exchange devices, so that the structure of the entire mixing equipment is not compact enough, and the heat exchange surface area is small, resulting in low heat exchange efficiency, which makes it inconvenient to carry out related operations and easily leads to safety accidents. Therefore, the traditional mixing device has the shortcomings of low stirring efficiency, high degree of danger, insufficient safety, large volume and insufficient compactness, and inability to effectively control the reaction temperature, and an effective solution needs to be proposed to solve them.

### SUMMARY

The purpose of the disclosure is to solve at least one of the technical problems existing in the field. Provided is a pressurized fluid mixing device, which can safely and efficiently mix two or more different fluids, or perform heat exchange and temperature control on more than one fluid, and has compact structure, greatly reduced space occupancy rate and large heat exchange surface area, thus improving the heat exchange efficiency.

The technical solutions that the disclosure adopts for solving its technical problem lie in:

a pressurized fluid mixing device, including an inner casing and an outer casing, wherein a first channel is arranged in the inner casing and comprises one or more unit channels, adjacent unit channels of which are communicated with each other, flow blocking members are fixed on the unit channels, the inner casing is provided with one or more first inlets and one or more first outlets, a second channel is arranged in the outer casing, the outer casing is provided with one or more second inlets and one or more second outlets, and the inner casing is fixed on the second channel.

In non-limiting embodiments or aspects, the inner casing has a shape of a long straight line, both ends of the inner casing extend out of the outer casing, and the inner casing is fixed to the outer casing in a sealing manner.

In non-limiting embodiments or aspects, the unit channels are superposed and connected laterally along a length direction of the inner casing, and the flow blocking members are cylindrical.

In non-limiting embodiments or aspects, a side wall of each of the unit channels and a side wall of each of the flow blocking members form a mixed flow channel with a cross section shaped as an ellipse, a circle, a polygon, a triangle or a wave shape.

In non-limiting embodiments or aspects, one or more first flow blocking teeth are fixed on the side wall of the flow blocking member, one or more second flow blocking teeth are fixed on an inner wall of the first channel, the first flow blocking teeth and the second flow blocking teeth are staggered, a first gap is formed between the first flow blocking teeth and the inner wall of the first channel, and a second gap is formed between the second flow blocking teeth and the side wall of the flow blocking member.

In non-limiting embodiments or aspects, one end of the flow blocking member is provided with a third channel passing through the flow blocking member and the inner casing, and the third channel is communicated with the second channel.

In non-limiting embodiments or aspects, the outer casing and the inner casing are made of metal, plastic or ceramic materials.

In non-limiting embodiments or aspects, the inner casing and the outer casing both have a thickness of 0.1 mm-5 mm; and the second channel has a volume which is 1-100 times a volume of the first channel.

In non-limiting embodiments or aspects, the first channel has a height of 0.5 mm-300 mm; and

each of the unit channels has a length of 3 mm-40 mm.

In non-limiting embodiments or aspects, the mixed flow channel has a width of 2 mm-40 mm; and

a excess gap is formed between the unit channels, the excess gap has a length of 0.05 mm-10 mm and a width of 1 mm-40 mm.

One of the above-mentioned technical solutions has the following beneficial effects. The pressurized fluid mixing device achieves the effects of efficient mixing and heat exchange by means of the organic combination of the inner casing and the outer casing. The inner casing is configured for transporting one or more fluids, and the first channel is arranged in the inner casing. By means of an external force, a pressure difference is generated between the fluids at the first inlet and the first outlet, which forces the fluids to pass through the first channel. The fluids pass through the flow blocking structure in the first channel to fully realize contact, mixing, collision, shearing, three-dimensional tumbling or reaction, thus improving the mixing and reaction efficiency between fluids. The second channel provided in the outer casing is configured for transporting a coolant or a heat preservation liquid, and the inner casing is fixed on the second channel. The coolant or heat preservation liquid directly acts on the outer wall of the inner casing, and is constantly renewed and flows, which increases the heat exchange surface area. The coolant can quickly transfer and exchange the mixed and reaction heat generated in the flow channel in time, so that the temperature of the inner cavity of the material flow channel can be effectively controlled, and the by-product and material degradation caused by the temperature increase can be avoided, thereby improving the safety of different mixing reactions. Moreover, during the transportation of the heat preservation liquid, the mixing cavity can be kept at a constant temperature, so that the fluids in the mixing cavity can be kept within a temperature range required for the reaction, which is beneficial to the advance of the reaction and improves the efficiency of fluid mixing reaction. Meanwhile, the solutions provided by the embodiments of the disclosure provide a simple, reliable and compact structure, occupy a small volume, and bring great convenience to the operation of the operator.

### BRIEF DESCRIPTION OF DRAWINGS

The disclosure is further described below in conjunction with accompanying drawings and non-limiting embodiments or aspects;

FIG. 1 is a schematic diagram of the overall structure of the first non-limiting embodiment or aspect of the disclosure;

FIG. 2 is a schematic diagram of the overall structure of the second non-limiting embodiment or aspect of the disclosure;

FIG. 3 is a schematic diagram of the overall structure of the third non-limiting embodiment or aspect of the disclosure;

FIG. 4 is a side sectional view of the first non-limiting embodiment or aspect of the disclosure;

FIG. 5 is a top sectional view of the first non-limiting embodiment or aspect of the disclosure;

FIG. 6 is an enlarged view of a part of the structure of the first non-limiting embodiment or aspect of the disclosure;

FIG. 7 is a top sectional view of the fourth non-limiting embodiment or aspect of the disclosure;

FIG. 8 is a top sectional view of the fifth non-limiting embodiment or aspect of the disclosure;

FIG. 9 is an enlarged view of a part of the structure of the fifth non-limiting embodiment or aspect of the disclosure;

FIG. 10 is a top sectional view of the sixth non-limiting embodiment or aspect of the disclosure;

FIG. 11 is an enlarged view of a part of the structure of the sixth non-limiting embodiment or aspect of the disclosure;

FIG. 12 is a top sectional view of the seventh non-limiting embodiment or aspect of the disclosure;

FIG. 13 is an enlarged view of a part of the structure of the seventh non-limiting embodiment or aspect of the disclosure;

FIG. 14 is a top sectional view of the eighth non-limiting embodiment or aspect of the disclosure;

FIG. 15 is an enlarged view of a part of the structure of the eighth non-limiting embodiment or aspect of the disclosure;

FIG. 16 is a top sectional view of the ninth non-limiting embodiment or aspect of the disclosure;

FIG. 17 is an enlarged view of a part of the structure of the ninth non-limiting embodiment or aspect of the disclosure;

FIG. 18 is a top sectional view of the tenth non-limiting embodiment or aspect of the disclosure;

FIG. 19 is an enlarged view of a part of the structure of the tenth non-limiting embodiment or aspect of the disclosure;

FIG. 20 is a top sectional view of the eleventh non-limiting embodiment or aspect of the disclosure;

FIG. 21 is a top sectional view of the twelfth non-limiting embodiment or aspect of the disclosure;

FIG. 22 is a top sectional view of the thirteenth non-limiting embodiment or aspect of the disclosure;

FIG. 23 is an enlarged view of a part of the structure of the thirteenth non-limiting embodiment or aspect of the disclosure;

FIG. 24 is a top sectional view of the fourteenth non-limiting embodiment or aspect of the disclosure; and

FIG. 25 is a top sectional view of the fifteenth non-limiting embodiment or aspect of the disclosure; and

### IN THE DRAWINGS

100 refers to inner casing; 110 refers to first inlet; 120 refers to first outlet; 130 refers to first channel; 140 refers to flow blocking member; 150 refers to mixed flow channel; 160 refers to first flow blocking tooth; 170 refers to second flow blocking tooth; 200 refers to outer casing; 210 refers to second inlet; 220 refers to second outlet; 230 refers to second channel; and 240 refers to third channel.

### DETAILED DESCRIPTION

This part will describe the specific embodiments of the disclosure in detail. The preferred embodiments of the disclosure are shown in the accompanying drawings, and the function of the drawings is to supplement the description of the text part with graphics, which enables people to intuitively and vividly understand each technical feature and the overall technical solutions of the disclosure, but cannot be understood as limiting the scope of protection of the disclosure.

In the description of the technical solutions of the disclosure, it should be understood that the positional descriptions referred to, for example, the directional or positional relationships indicated by up, down, front, rear, left, right, etc., are based on the directional or positional relationships shown in the drawings, and are only for convenience and simplification of description of the disclosure, but not for indicating or implying that the referred device or element must have a specific direction, be constructed and operated in a specific direction, and thus should not be construed as limiting the disclosure.

In the description of the technical solutions of the disclosure, “several” means one or more, “a plurality of” means more than two, “greater than a number”, “less than a number”, “exceed a number” and the like indicate that the number is excluded, and “above a number”, “below a number”, “within a number”, and the like indicate that the number is included. “First” and “second” are only used to distinguish between technical features but cannot be used to indicate or imply relative importance or implicitly specify a quantity of indicated technical features or implicitly specify a sequential relationship of indicated technical features.

In the description of the technical solutions of the disclosure, unless otherwise expressly defined, the terms such as “disposed”, “mounted”, and “connected” should be understood in a broad sense. For persons of ordinary skill in the art, specific meanings of the terms in the disclosure may be appropriately determined with reference to the specific content in the technical solution.

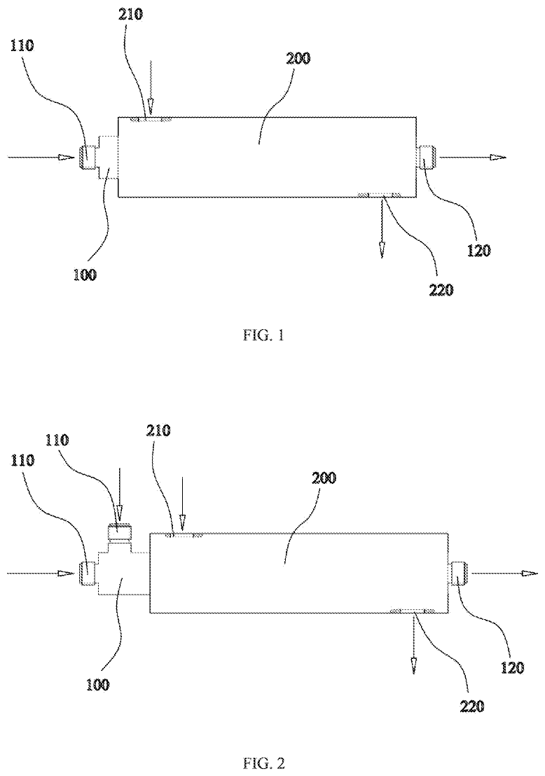

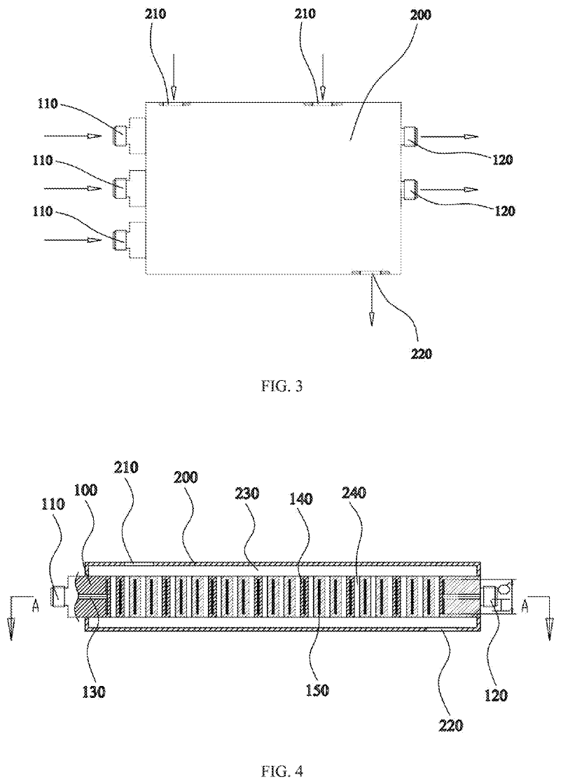

Referring to FIGS. 1-5, a pressurized fluid mixing device, including an inner casing 100 and an outer casing 200, wherein a first channel 130 is arranged in the inner casing 100 and includes one or more unit channels, adjacent unit channels of which are communicated with each other, flow blocking members 140 are fixed on the unit channels, the inner casing 100 is provided with one or more first inlets 110 and one or more first outlets 120, a second channel 230 is arranged in the outer casing 200, the outer casing 200 is provided with one or more second inlets 210 and one or more second outlets 220, and the inner casing 100 is fixed on the second channel 230.

In the non-limiting embodiments or aspects mentioned above, specifically, the first channel 130 provided in the inner casing 100 is configured for transporting one or more pressurized fluids. The shape of the flow blocking members is selected according to actual needs, and the flow blocking structure may be designed as a plate shape, also a column shape, or a comprehensive application of a plate body and a column body, with the purpose of making the fluids generate irregular turbulences when flowing in the first channel 130, so as to improve the mixing or reaction effect, thereby improving the mixing or reaction efficiency. The fluids to be mixed or reacted enter from the first inlet 110, and are fully mixed, sheared, contacted and collided in the first channel 130 by means of the flow blocking structure, so that the materials can be fully contacted to achieve a high-efficiency mixing and reaction effect, and the mixing effect is close to the effect of a traditional stirring tank stirring at 3000 rpm. The fluids finally flow out from the first outlet 120. Since there are a variety of fluids with different properties in the fluids to be mixed or the fluids to be reacted, one or more first inlets 110 may be provided. If there is one first inlet designed, the fluids may be first subjected to initial mixing from the outside, and then injected into the first channel 130 through the first inlet 110 in a pressurized manner, so as to perform deep and efficient mixing. If there are a plurality of first inlets designed, each of the fluids may be injected from a respective first inlet 110, and then be mixed and reacted in the first channel 130 at one time, and finally the finished fluid flows out from the first outlet 120. Compared with the traditional mixing and stirring tanks, stirring towers, etc., the above two cases have the advantages of continuous, efficient and stable mixing and reaction.

The second channel 230 is arranged in the outer casing 200, and the inner casing 100 is fixed in the second channel 230. A coolant or a heat preservation liquid etc. may flow in the second channel 230 according to the actual task requirements. When being injected into the second channel 230, the coolant may directly act on the outer wall of the inner casing 100 to increase the heat exchange area, and continuously flow and be renewed on the outer wall of the inner casing 100, so that the heat generated by mixing and reaction in the inner casing 100 can be transferred in time through heat exchange, the heat exchange efficiency is improved, and further, the temperature in the first channel 130 can be effectively controlled. The continuous delivery of the coolant avoids the by-products and material degradation due to the temperature increase, and also avoids some safety hazards caused by excessive temperature. It can be seen that the device in the disclosure has higher safety than traditional stirring reactors, reaction towers, etc., and also reduces the space occupancy rate of the device itself, making it compact in structure to be convenient for production and operator to operate and use. If the heat preservation liquid is transported in the second channel 230, the mixing cavity can be kept in a constant temperature state, so that the fluids in the mixing cavity can be kept within a temperature range required for the reaction, which is conducive to the advance of the reaction and improves the efficient of fluid mixing reaction.

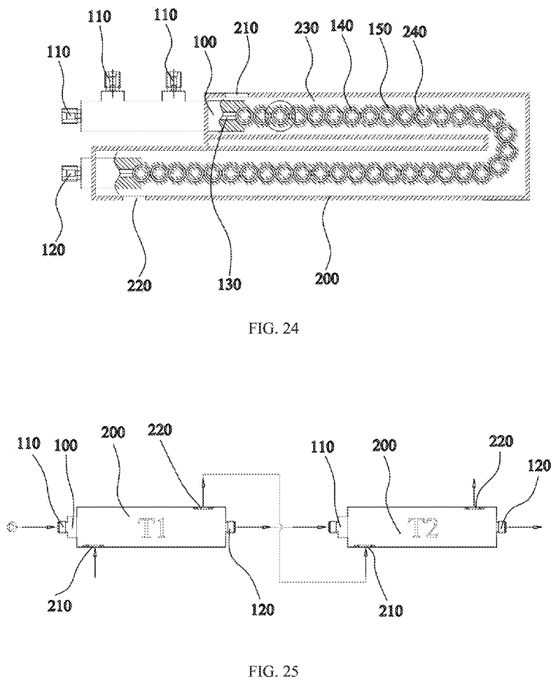

Further, the inner casing 100 has a shape of a long straight line, both ends of the inner casing 100 extend out of the outer casing 200, and the inner casing 100 is fixed to the outer casing 200 in a sealing manner. Specifically, the inner casing 100 in the shape of a long straight line is convenient for production and assembly on the one hand, and on the other hand, improves the compactness of the device and facilitates the installation of the device by the operator. Both ends of the inner casing 100 extend out of the outer casing 200, which is beneficial to provide the first inlet 110 and the first outlet 120 at the extension part, and also beneficial to inject the fluids to be mixed in a pressurized manner. The sealing portion between the inner casing 100 and the outer casing 200 may be fixed by welding, or be quickly installed and fixed by means of an industrial sealant, or be fixed by means of integral molding and fixtures. Meanwhile, the shape of the inner casing 100 may also be non-linear, such as the U shape in FIG. 24, and the U-shaped design can increase the flow stroke in the first channel 130 without increasing the overall lateral length of the inner casing 100, thus improving the mixing or reaction effect on the premise of keeping the structure compact.

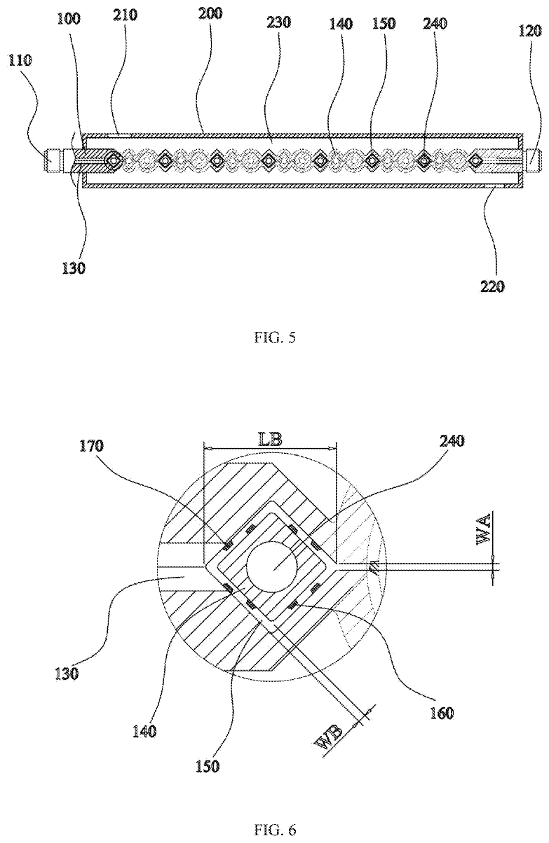

Further, referring to FIGS. 4 and 5, the unit channels are superposed and connected laterally along a length direction of the inner casing 100, and the flow blocking members are cylindrical. Specifically, the lateral superposition and connection of the unit channels along the inner casing 100 is a preferred solution that makes the structure of the inner casing 100 more compact. Certainly, the unit channels may also be selectively designed in an S-shaped distribution, a Z-shaped distribution, etc. in the inner casing. Moreover, the unit channels may also be designed into a variety of different shapes. In addition to a column shape, the flow blocking members 140 may also be designed into a variety of different shapes to increase the irregularity of turbulences formed in the flow of fluids in the first channel 130, so as to improve the mixing and shearing effect.

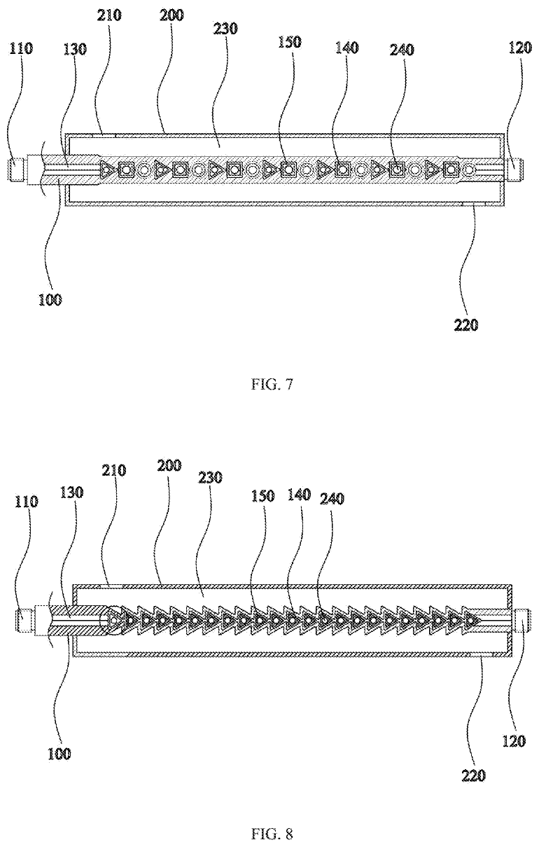

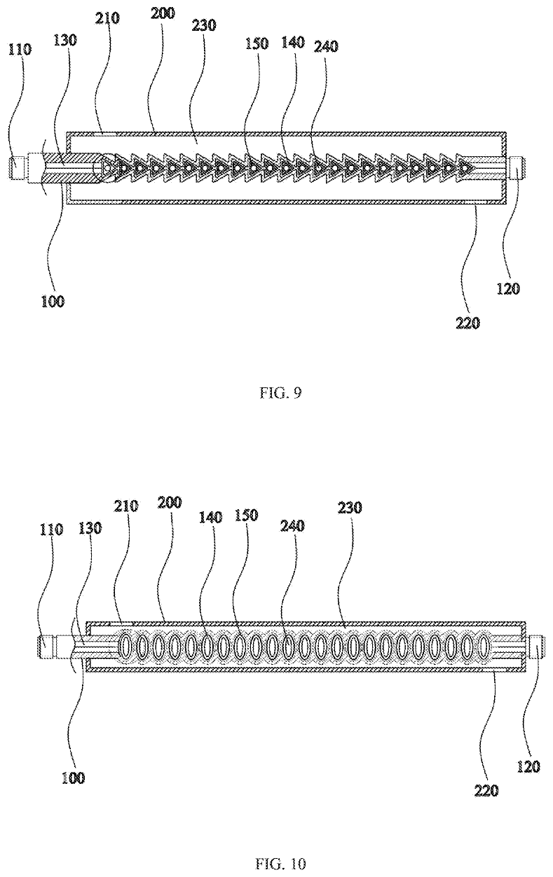

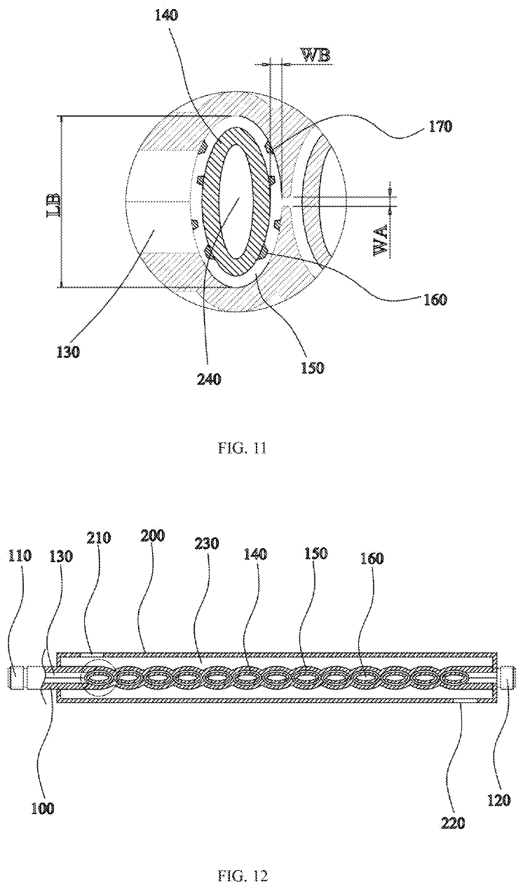

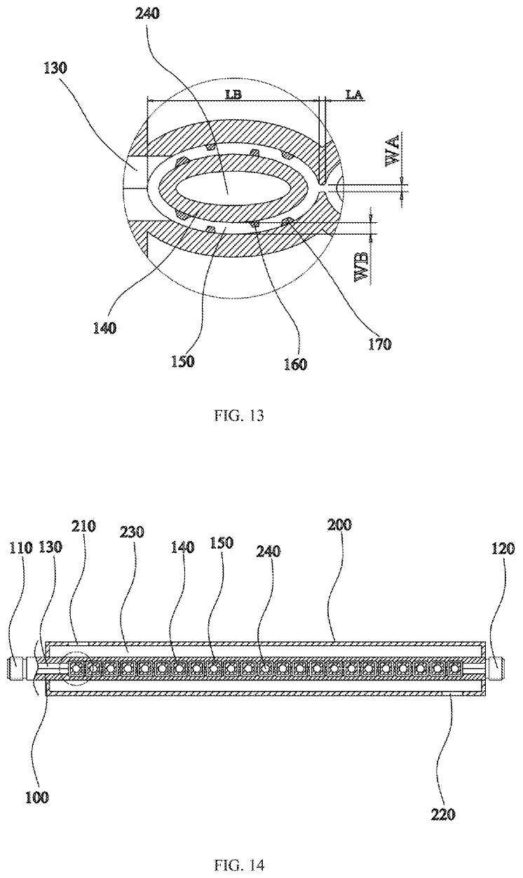

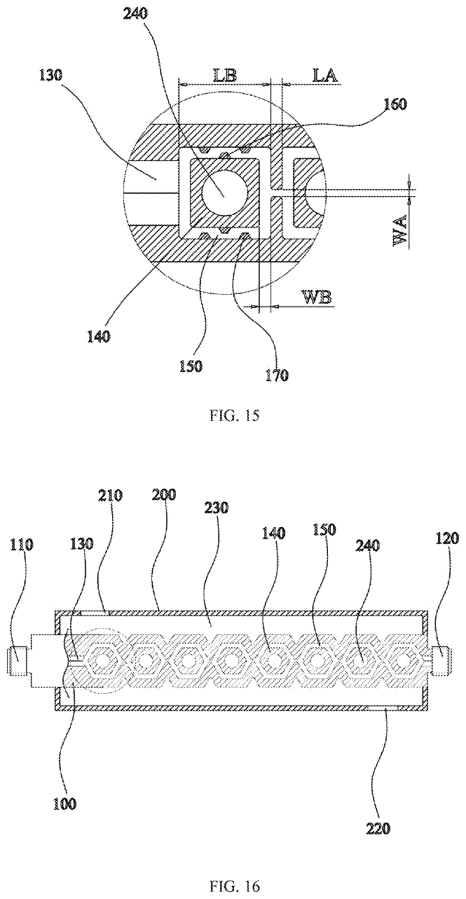

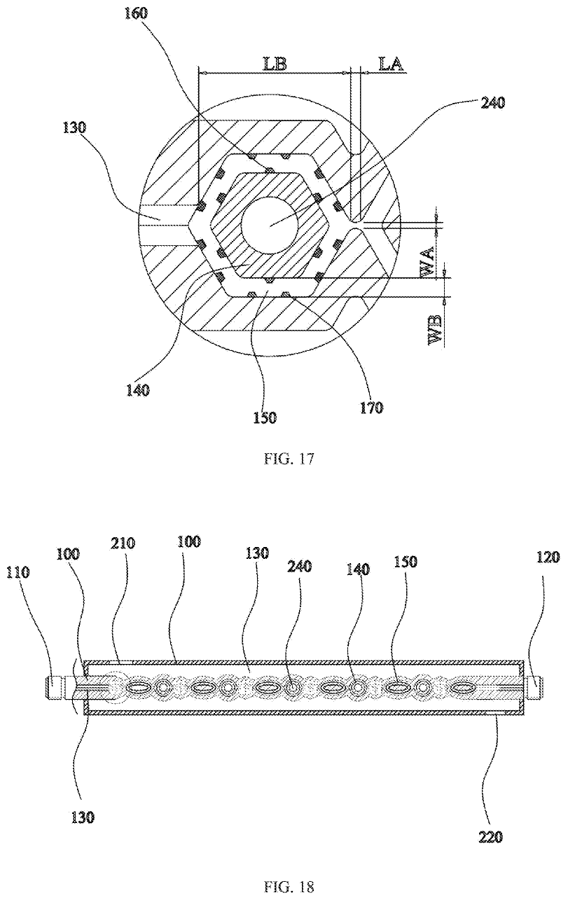

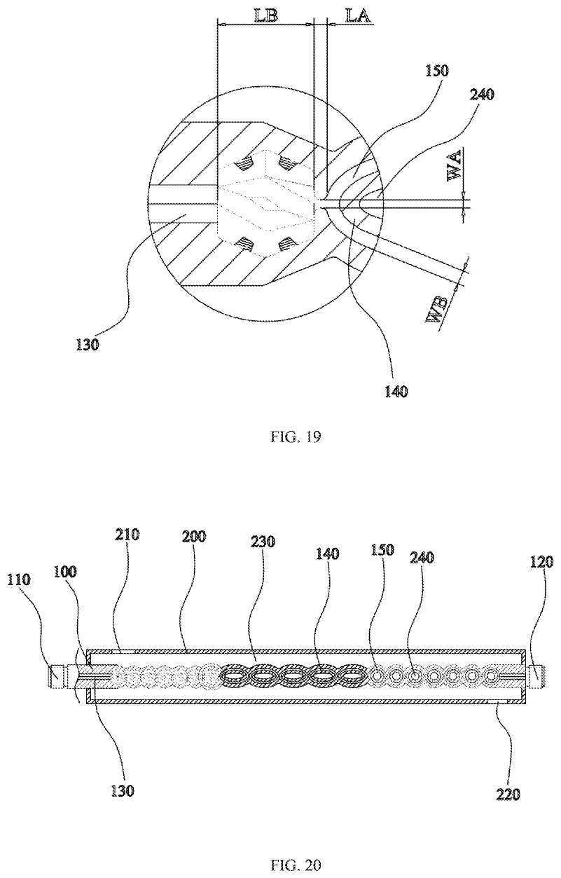

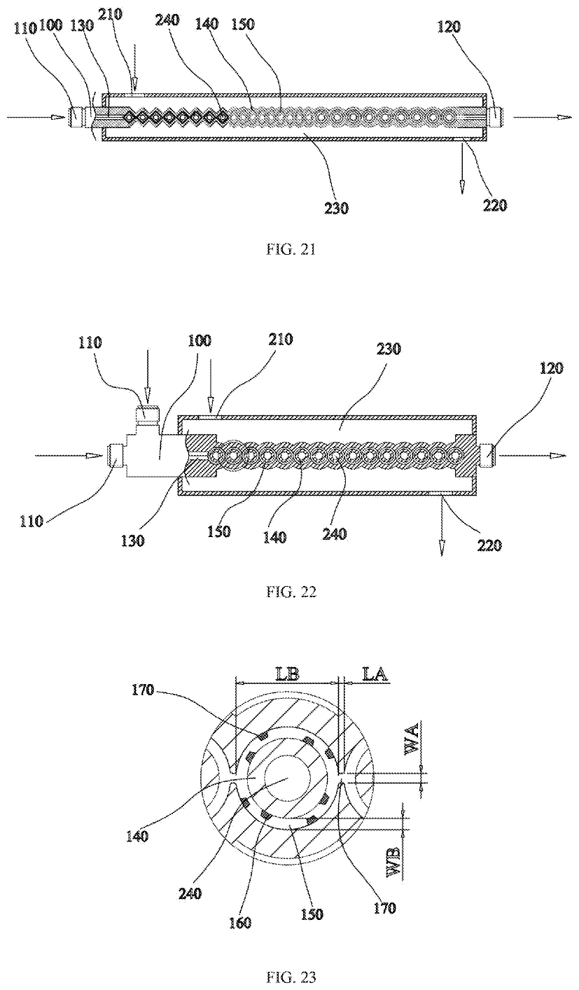

Further, referring to FIGS. 5-23, the side walls of the unit channels and the side walls of the flow blocking members 140 form a mixed flow channel 150, and the cross-sectional shape of the mixed flow channel 150 includes one or more of an ellipse, a circle, a polygon, a triangle or a wave shape. Specifically, in the pressurized fluid mixing device with a heat exchange function provided in the present embodiment, the shapes of the unit channels and the flow blocking members 140 are designed to be consistent, so that the side walls of the unit channels and the side walls of the flow blocking members 140 form a mixed flow channel 150 of a fixed size. Therefore, the cross-sectional shape presented by the mixed flow channel 150 is related to the specific shapes of the unit channels and the flow blocking members 140. In addition to the shapes included in the above-mentioned non-limiting embodiments or aspects, an L-shape, a V-shape, a U-shape, an Σ-shape etc. may also be used. Mixed flow channels 150 of various shapes may be freely combined and arranged according to the properties of actually transported fluids, so that the top view section of the mixed flow channels 150 presents a diverse and complicated structure, so as to achieve the best mixing and reaction effect.

Further, referring to FIG. 6, one or more first flow blocking teeth 160 are fixed on the side walls of the flow blocking members 140, one or more second flow blocking teeth 170 are fixed on the inner wall of the first channel 130, the first flow blocking teeth 160 and the second flow blocking teeth 170 are staggered, a first gap is formed between the first flow blocking teeth 160 and the inner wall of the first channel 130, and a second gap is formed between the second flow blocking teeth 170 and the side walls of the flow blocking members 140. Specifically, the first gap and the second gap are provided to further increase the mixed shear strength of different fluids, and the staggered distribution of the first flow blocking teeth 160 and the second flow blocking teeth 170 enables the fluids to form irregular turbulences after passing through the first gap and the shear gap, which are repeatedly mixed and sheared with the subsequently passing fluids, so that full mixing or reaction of different fluids is promoted, and the mixing or reaction rate is improved. On the other hand, the flow blocking teeth also play the role of reinforcing ribs, which help to improve the structural strength of the flow blocking elements.

Further, referring to FIGS. 4-6, one end of the flow blocking member 140 is provided with a third channel 240 passing through the flow blocking member 140 and the inner casing 100, and the third channel 240 is communicated with the second channel 230. Specifically, the third channel 240 can allow a coolant or a heat preservation liquid to pass through to further increase the heat exchange surface area of the device provided in the present embodiment, so as to further improve the heat exchange efficiency of the mixed or reaction fluids, and at the same time improve the flow rate of the coolant or the heat preservation liquid, thus enhancing the cooling or heat preservation effect on the inner casing.

Further, the outer casing 200 and the inner casing 100 are made of metal, plastic or ceramic materials, such as titanium, zirconium, tantalum, PTFE, PEEK, carbon fiber, glass, carbon steel, C4 stainless steel, 2205 double molybdenum stainless steel, nickel-based 625 stainless steel, Hastelloy C276, Hastelloy B, Hastelloy C2000, PET, zirconia, silicon nitride, silicon carbide. Specifically, the material composition of the inner casing 100 and the outer casing 200 may be determined according to the specific properties of the fluids. When the inner casing 100 and the outer casing 200 are designed to be made of metal, a 3D printer for metal may be used for production, which can meet the precision of the first channel 130 and the second channel 230, so that the size of the first channel 130 and the second channel 230 can be strictly controlled, and the first channel 130 and the second channel 230 can obtain a strong pressure bearing capacity, and the structural stability of the inner casing 100 and the outer casing 200 can be improved, thus improving the overall safety of the device provided in the present embodiment. When the inner casing 100 and the outer casing 200 are made of lightweight plastic materials, the inner casing 100 and the outer casing 200 can be applied to task requirements of fluids with small quantity or small incident pressure. The device body made of lightweight plastic, although with smaller pressure-bearing capacity than metal materials, is easy to carry and transport, and also convenient for operator to install and operate. When the inner casing 100 and the outer casing 200 are designed to be made of ceramic material, it is suitable to make the first channel 130 and the second channel 230 with large volume, to mix the fluids with high throughput. The ceramic material itself has the characteristic of high strength, so that the device provided in the present embodiment has strong pressure bearing capacity and is not easy to be corroded by the fluids, which prevents the fluids from causing great damage to the device provided in the present embodiment, and improves the service life of the device.

Further, referring to FIGS. 4, 5 and 6, the wall thicknesses of the inner casing 100 and the outer casing 200 are both 0.1 mm-5 mm;

the volume of the second channel 230 is 1-100 times that of the first channel 130;

the height of the first channel 130 corresponds to Ha in FIG. 4, with its range being 0.5 mm-300 mm;

the length of the unit channels corresponds to LB in FIG. 6, with its range being 3 mm-40 mm;

the width of the mixed flow channel 150 refers to the interval between the side walls of the unit channels and the side walls of the flow blocking elements 140, that is, WB in FIG. 6, with its range being 2 mm-40 mm; and

excessive gaps are formed between the unit channels, the length of the excessive gaps corresponds to LA in FIG. 6, with its range being 0.05 mm-10 mm, and the width of the excessive gaps corresponds to WA in FIG. 6, with its range being 1 mm-40 mm.

Specifically, when the wall thicknesses of the inner casing 100 and the outer casing 200 are 0.1 mm, the height Ha of the first channel 130 is 0.5 mm, the length LB of the unit channels is 3 mm, the width WB of the mixed flow channel 150 is 2 mm, the length LA and the width WA of the excessive gaps are both 1 mm, the volume of the second channel 230 is 10 times that of the first channel 130, and the inner casing 100 and the outer casing 200 are made of nickel 625 stainless steel, which can adapt to the fluid bearing pressure of about 0.6 Mpa and is configured for conveying and mixing the fluids with small flow.

When the wall thicknesses of the inner casing 100 and the outer casing 200 are 5 mm, the height Ha of the first channel 130 is 300 mm, the length LB of the unit channels is 40 mm, the width WB of the mixed flow channel 150 is 40 mm, the length LA of the excessive gaps is 10 mm, the width WA of the excessive gaps is 40 mm, the volume of the second channel 230 is 100 times that of the first channel 130, and the inner casing 100 and the outer casing 200 are made of nickel 625 stainless steel, which can adapt to the fluid bearing pressure of about 40 Mpa, and is configured for conveying and mixing the fluids with large flow.

When the wall thicknesses of the inner casing 100 and the outer casing 200 are 2 mm, the height Ha of the first channel 130 is 100 mm, the length of the unit channels is 20 mm, the width WB of the mixed flow channel 150 is 20 mm, the length LA of the excessive gaps is 5 mm, the width WA of the excessive gaps is 20 mm, the volume of the second channel 230 is 30 times that of the first channel 130, and the inner casing 100 and the outer casing 200 are made of nickel 625 stainless steel, which can adapt to the fluid bearing pressure of about 25 Mpa, and is configured for conveying and mixing the fluids with middle flow.

The pressurized fluid mixing device according to an embodiment of the disclosure can be configured for mixing, shearing, heat exchange and reaction between different gases, liquids, solid-liquid mixtures and powders in chemical, food, daily chemical, petrochemical, fine chemical and other industries; and its mixing, reaction and heat exchange types are not limited to nitration, sulfonation, chlorination, hydrogenation, diazotization, condensation, acylation, esterification, transposition, fluorination, amination, peroxidation, hydrogenation, polymerization, cracking, oximation and neutralization.

The pressurized fluid mixing device according to an embodiment of the disclosure can be produced by means of manufacturing methods such as solid casting, 3D printing, welding, high-temperature diffusion welding, screws, and fixture fixing, etc. In practical applications, by taking a conventional metal printer as an example, after the steps such as model design, model repair, placement and slicing, the set parameters are: laser spot: 100 um; scanning speed: 966 mm/s; scanning distance: 0.1 mm; and particle size: 15-53 um, and the material used is nickel-based 625 stainless steel. The product according to an embodiment of the disclosure can be printed, with its bearing pressure reaching 40 Mpa, and its operating temperature being −100° C. to 500° C.

In practical applications, toluene of 200 ml/min as a first fluid and water of 100 ml/min as a second fluid respectively enter into the device according to an embodiment of the disclosure from the inlet, there is one device, the total stroke of the first channel 130 is 250 mm, and the pressure is 0.3-0.6 Mpa. After the two fluids are mixed, 95% is emulsified, and the mixing effect is excellent.

In practical applications, the chemical raw material A is mixture of nitric acid and sulfuric acid with a flow rate of 50 ml/min, and the chemical raw material B has a flow rate of 20 ml/min. At room temperature of 30° C., and the chemical raw materials A and B pass through the device according to an embodiment of the disclosure. At the same time, a coolant of −10° C. is introduced into the second channel 230 to control the reaction temperature. The reaction temperature is 40° C., the residence time lasts 3 seconds, and the nitrification is completed, with the main yield content of 98% and the nitrification raw material B remaining 0.2%. The present reaction realizes the safe production of nitrification.

In addition, referring to FIG. 25, a plurality of pressurized fluid mixing devices provided by the disclosure can be arranged to form a mixed reaction system to further improve the mixing effect of fluids. Taking a whole system composed of two devices as an example, the fluids to be reacted flow through the first inlet 110 of the T1 device in FIG. 25 and are mixed and reacted in the mixing cavity in the T1 device, and can flow to the first inlet 110 of the T2 device through a pipeline after flowing out from the first outlet 120 of T1, so that the heat generated by mixing the reaction liquids is further transferred by means of heat exchange while increasing the mixing process at the same time. Finally, when flowing out from T2, the reaction liquids have been fully mixed, the temperature required by production tasks can also be maintained, and the coolant or the heat preservation liquid flows in the second channels 230 of the T1 device and the T2 device, and flows, from the second outlet 220 of T1, to the second inlet 210 of T2 via a connecting pipeline. In addition, the internal structures of the device T1 and the device T2 may be different, and the top-view cross-sectional shape of the mixed flow channels 150 can also be arbitrarily designed and arranged. Such freely combined modular system can flexibly respond to a variety of complicated mixing task requirements, and the effect of mixing and heat exchange is unmatched by traditional reactors and reaction towers.

In practical applications, the chemical raw material A of formaldehyde with a flow rate of 750 ml/min as a first fluid, the chemical raw material B of butyraldehyde with a flow rate of 690 ml/min as a second fluid, and the chemical raw material C of alkali water with a flow rate of 750 ml/min as a third fluid respectively enter the device according to an embodiment of the disclosure from a feed nozzle, there are four devices, the total stroke of the first channel 130 is 1000 mm, the pressure is 0.6 Mpa, constant temperature is kept with hot water, the constant temperature is 70° C., and the temperature at the material reaction outlet is 55° C. After passing through the device according to an embodiment of the disclosure, which lasts for 10 seconds, the reactions are all completed.

In practical applications, the raw materials, which are a corn oil fluid A containing an emulsifier and a water fluid B, are subjected to an emulsification experiment. After passing through two devices according to an embodiment of the disclosure, in which the total stroke of the first channel 130 is 500 mm, and the flow rates are 100 L/min for fluid A and 200 ml/min for fluid B, a water emulsion product is obtained at the outlet, and the particle size of the water emulsion is 1.5 um after analysis, which achieves the same effect as a traditional high-efficiency shearing machine.

In practical applications, diclofenac acid chloride used as material A and a tetrafluorobenzyl alcohol toluene solution used as material B are subjected to an esterification reaction. After passing through four devices according to an embodiment of the disclosure, in which the total stroke of the first channel 130 is 1000 mm, the flow rates are 100 L/min for fluid A and 400 ml/min for fluid B, the temperature is controlled with constant temperature water at 40-80° C., and the residence time lasts for 10 seconds, a 99% tetrafluthrin toluene liquid product is obtained at the outlet. Compared with a three-necked bottle dropping synthesis method, the production time is shortened by 1 hour, and 98% of the production time is saved; and

In practical applications, the metered raw material solution contains a beta-cypermethrin solution A, an emulsifier B and deionized water C, and is slightly stirred with a stirring speed below 100 rpm. After passing through four devices according to an embodiment of the disclosure using a metering pump, in which the total stroke of the first channel 130 is 1000 mm, the temperature is controlled below 10° C. with constant temperature water, and the residence time lasts for 10 seconds, a beta-cypermethrin water emulsion is obtained. By comparison, the shearing effect reaches that of using a shearing machine of 1500 rpm/min for 60 minutes, which improves production efficiency and reduces energy consumption.

The non-limiting embodiments or aspects of the disclosure have been described in detail above in conjunction with the accompanying drawings, but the disclosure is not limited to the above-mentioned non-limiting embodiments or aspects, and within the scope of knowledge possessed by those of ordinary skill in the art, various changes can also be made without departing from the purpose of the disclosure.

1. A pressurized fluid mixing device, comprising an inner casing and an outer casing, wherein:
a first channel is arranged in the inner casing and comprises one or more unit channels, adjacent unit channels of which are communicated with each other, flow blocking members are fixed on the unit channels, the inner casing is provided with one or more first inlets and one or more first outlets, a second channel is arranged in the outer casing, the outer casing is provided with one or more second inlets and one or more second outlets, and the inner casing is fixed on the second channel.

2. The pressurized fluid mixing device of claim 1, wherein:
the inner casing has a shape of a long straight line, both ends of the inner casing extend out of the outer casing, and the inner casing is fixed to the outer casing in a sealing manner.

3. The pressurized fluid mixing device of claim 2, wherein:
the unit channels are superposed and connected laterally along a length direction of the inner casing, and the flow blocking members are cylindrical.

4. The pressurized fluid mixing device of claim 3, wherein:
a side wall of each of the unit channels and a side wall of each of the flow blocking members form a mixed flow channel with a cross section shaped as at least one of the following: an ellipse, a circle, a polygon, a triangle, a wave shape, or any combination thereof.

5. The pressurized fluid mixing device of claim 3, wherein:
one or more first flow blocking teeth are fixed on the side wall of the flow blocking member, one or more second flow blocking teeth are fixed on an inner wall of the first channel, the first flow blocking teeth and the second flow blocking teeth are staggered, a first gap is formed between the first flow blocking teeth and the inner wall of the first channel, and a second gap is formed between the second flow blocking teeth and the side wall of the flow blocking member.

6. The pressurized fluid mixing device of claim 3, wherein:
one end of the flow blocking member is provided with a third channel passing through the flow blocking member and the inner casing, and the third channel is communicated with the second channel.

7. The pressurized fluid mixing device of claim 1, wherein:
the outer casing and the inner casing are made of at least one of the following: metal, plastic, ceramic materials, or any combination thereof.

8. The pressurized fluid mixing device of claim 1, wherein:
the inner casing and the outer casing both have a thickness of 0.1 mm-5 mm; and
the second channel has a volume which is 1-100 times a volume of the first channel.

9. The pressurized fluid mixing device of claim 1, wherein:
the first channel has a height of 0.5 mm-300 mm; and
each of the unit channels has a length of 3 mm-40 mm.

10. The pressurized fluid mixing device of claim 4, wherein:
the mixed flow channel has a width of 2 mm-40 mm; and
a excess gap is formed between the unit channels, the excess gap has a length of 0.05 mm-10 mm and a width of 1 mm-40 mm.
